(19) United States
(12) Patent Application Publication
(10) Pub. No.: US 2016/0331213 A1
(43) Pub. Date: Nov. 17, 2016

(54) PORTABLE INSPECTION SYSTEM

(71) Applicant: OPTIMEDE INC., Seoul (KR)

(72) Inventor: Hun Tae KIM, Seoul (KR)

(21) Appl. No.: 15/033,208

(22) PCT Filed: Oct. 31, 2014
(86) PCT No.: PCT/KR2014/010340

§ 371 Date: Jul. 15, 2016

(30) Foreign Application Priority Data
Oct. 31, 2013 (KR) 10-2013-0131128
Oct. 31, 2013 (KR) 10-2013-0131129
Oct. 31, 2013 (KR) 10-2013-0131130

(51) Int. Cl. A61B 1/00; A61B 1/005; A61B 1/045; A61B 5/00; A61B 1/05; A61B 1/06

(52) U.S. Cl. CPC A61B 1/00108; A61B 1/05; A61B 1/0676; A61B 1/00052; A61B 1/00041; A61B 1/00124; A61B 1/00006; A61B 1/00029; A61B 1/045; A61B 1/0002; A61B 1/0005; A61B 5/6898; A61B 1/005

(57) ABSTRACT

Provided is a portable endoscope system. The portable endoscope system includes: an endoscope including a terminal end at which a camera and a lighting device are provided, a head provided on the opposite side to the terminal end, an actuator provided within the head to bend the terminal end, and a controller provided within the head to control the actuator, the camera, and the lighting device; and an intelligent portable device including a touch screen, loaded with an endoscope driving application, and provided to be electrically connected to the controller.

### TECHNICAL FIELD

The present disclosure relates to a portable inspection system, and more particularly, to a portable inspection system which an endoscopy operator can easily carry about. Further, the present disclosure relates to an inspection system capable of marking a defect in real time by directly indicating a defect of an inspection target with a marker while inspecting the inspection target. Furthermore, the present disclosure relates to a terminal end steering device of an endoscope device.

### BACKGROUND

Nowadays, with the rapid advancement of technology, inspection systems capable of inspecting whether there is a defect inside or outside an inspection target are being developed and applied to various industrial fields. For example, a medical endoscope system is being applied to a medical field in order to inspect whether there is a defect (or lesion) within organs of a human body, and an industrial endoscope system is being applied in order to inspect whether there is a defect within various kinds of industrial apparatuses.

Conventionally, in a process of inspecting an inspection target using such an inspection system, if defects are found, an inspector stores images respectively showing the defects, and the inspector checks the stored images in sequence while marking every defect shown in the images after the inspection. However, according to this inspection system, the inspector needs to perform an operation for inspecting the inspection target and an operation for marking a defect of the inspection target which are separated in time, which causes great hassle to the inspector.

A technology which can solve the above-described problem is known. This gist of this technology is about indicating a defect with a physical marker when the defect is found while inspecting an inspection target. One example of this technology is disclosed in Korean Patent Laid-open Publication No. 10-2006-0036080. According to Korean Patent Laid-open Publication No. 10-2006-0036080, a capsule endoscope inserted into a human body is configured to spray a liquid marker to a lesion site in response to a signal received from the outside.

Further, endoscopes are roughly classified into medical endoscopes and industrial endoscopes depending on where they are used. A medical endoscope is a medical device designed to directly observe and treat a lesion site of an organ without a surgery or an autopsy. Further, an industrial endoscope is a device designed to observe the inside of an apparatus without the need to disassemble the apparatus in various industrial fields.

An endoscope system includes an endoscope, an image processing device, and other peripheral devices. The image processing device is configured to process an image captured by a camera provided at a terminal end of the endoscope to visually show the image to a user. Examples of this technology are disclosed in Korean Patent Laid-open Publication No. 10-2007-0104539, Korean Patent Laid-open Publication No. 10-2012-0133182, and Korean Utility Model Laid-open Publication No. 20-2012-0001758.

The image processing device digitally processes an image. The digitally-operated image processing device is bulky and heavy and thus almost impossible to carry about. Therefore, it is very difficult to use the endoscope system in other places than a specific place where the endoscope system is installed. Further, the digitally-operated image processing device is very expensive.

Furthermore, generally, at a terminal end of an endoscope, a camera configured to photograph a lesion site or the inside of an apparatus and a lighting device configured to irradiate a light to the lesion site or the inside of the apparatus are provided. Moreover, a medical endoscope includes a hole-shaped channel at a terminal end. A surgical device such as forceps or a laser irradiator passes through the channel.

In order to accurately observe the lesion site or the inside of the apparatus, the camera and the lighting device need to be variously changed in direction. Therefore, the terminal end of the endoscope is designed to be freely bendable in various directions, and the endoscope includes a steering device configured to adjust a direction of the terminal end. Examples of this technology are disclosed in Korean Patent Laid-open Publication No. 10-2007-0104539, Korean Patent Laid-open Publication No. 10-2012-0133182, and Korean Utility Model Laid-open Publication No. 20-2012-0001758.

### DISCLOSURE OF THE INVENTION

### Problems to be Solved by the Invention

According to a conventional technology of indicating a defect of an inspection target with a physical marker as disclosed in Korean Patent Laid-open Publication No. 10-2006-0036080, while an inspection target is inspected, a defect in the inspection target may be indicated with a marker. However, according to the conventional technology, a physical marker, a mechanical means for indicating the defect of the inspection target with the physical marker, and an electronic means for controlling the mechanical means are needed.

Accordingly, the present disclosure provides an inspection system which can perform an operation of marking a defect of an inspection target in real time while inspecting the inspection target and does not need a physical marker for this operation.

Further, the present disclosure is conceived to solve the problem of a conventional endoscope system. Thus, the present disclosure provides a portable endoscope system which is cheap and very easy to carry about.

Furthermore, the present disclosure provides a terminal end steering device configured to adjust a bending direction of a terminal end, such as a terminal end of an endoscope, designed to be bendable in various directions. Moreover, the present disclosure provides an endoscope including the terminal end steering device.

### Means for Solving the Problems

According to an aspect of the present disclosure, there is provided a portable endoscope system including: an endoscope including a terminal end at which a camera and a lighting device are provided, a head provided on the opposite side to the terminal end, an actuator provided within the head to bend the terminal end, and a controller provided within the head to control the actuator, the camera, and the lighting device; and an intelligent portable device including a touch screen, loaded with an endoscope driving application, and provided to be electrically connected to the controller.

At the head of the endoscope, a cradle for mounting the intelligent portable device thereon is provided.

After the endoscope and the intelligent portable device are electrically connected to each other and the endoscope driving application is executed, the intelligent portable device supplies the controller with power required to operate the actuator, the camera, the lighting device, and the controller.

Further, after the endoscope and the intelligent portable device are electrically connected to each other and the endoscope driving application is executed, the intelligent portable device receives a current image being captured by the camera from the controller and displays the current image on the touch screen.

Herein, the intelligent portable device displays a zoom-in/out icon for zooming in to and out from the current image together with the current image on the touch screen, and if the zoom-in/out icon is touched, the intelligent portable device transmits a zoom-in/out signal corresponding thereto to the controller.

Further, if the current image is dragged, the intelligent portable device transmits terminal bending signal, which instructs the controller to bend the terminal end of the endoscope in a dragged direction, to the controller.

Furthermore, if any one point in the current image is touched, the intelligent portable device generates a marker at the touched point and then captures the current image including the marker and stores the current image in its memory.

Moreover, the intelligent portable device displays a stored image read from a folder specified by a user together with the current image on the touch screen.

Further, the intelligent portable device displays a page turn icon together with the stored image on the touch screen, and if the page turn icon is touched, the intelligent portable device displays another stored image in the folder together with the current image on the touch screen.

Furthermore, the intelligent portable device displays an on/off icon for the lighting device together with the current image on the touch screen, and if the on/off icon is touched, the intelligent portable device transmits an on/off signal corresponding thereto to the controller.

Moreover, the intelligent portable device displays an on/off icon for a recorder included therein together with the current image on the touch screen, and if the on/off icon is touched, the intelligent portable device operates or stops the recorder.

According to another aspect of the present disclosure, there is provided an inspection system capable of marking a defect in real time, including: an image acquisition device configured to acquire a current inspection image of an inspection target; and an intelligent device including a touch screen and configured to receive the current inspection image from the image acquisition device and display the current inspection image on the touch screen. Herein, if any one point in the current inspection image is touched, the intelligent device indicates the touched point with a marker and then captures and stores the current inspection image including the marker in a memory.

The intelligent device assigns an identifier to the marker and then displays the marker on the touch screen.

The intelligent device displays a file open icon together with the current inspection image on the touch screen, and displays a past inspection image specified by an inspector through a touch on the file open icon together with the current inspection image on the touch screen.

The intelligent device displays a file turn icon together with the past inspection image on the touch screen, and if the file turn icon is touched, the intelligent device displays a previous or next past inspection image in the folder including the past inspection image together with the current inspection image on the touch screen.

The image acquisition device is manufactured into a tube shape (e.g., endoscope) to be inserted into the inspection target without a need to disassemble or incise the inspection target. In this case, desirably, the intelligent device may be a smart phone or a smart pad, and desirably, the image acquisition device may include a cradle for mounting the smart phone or the smart pad thereon.

According to yet another aspect of the present disclosure, there is provided a terminal end steering device configured to adjust a direction of a terminal end designed to be bendable. The terminal end steering device includes: a first wire configured to bend the terminal end in a forward direction while pulling one side of the terminal end during a forward operation of a first driving means; a second wire configured to bend the terminal end in a backward direction while pulling the other side of the terminal end during a forward operation of a second driving means; a switching means mounted on the first wire and the second wire and configured to output an electrical signal only when the terminal end is bent in the forward direction and the backward direction; and a control unit configured to operate any one driving means, which does not operate in the forward direction, of the first driving means and the second driving means in the backward direction when the electrical signal is output from the switching means.

Each of the first wire and the second wire is separated by the switching means into a lower wire located on the side of the terminal end and an upper wire located on the opposite side to the lower wire, and the switching means includes: a case connected to the upper wire; a sliding plate slidably accommodated within the case and connected to the lower wire; a tensile spring accommodated within the case and including both ends respectively combined with case and the sliding plate; and a touch sensor mounted on at least one of the case and the sliding plate.

Otherwise, the switching means may include a torque spring including one end connected to the lower wire and the other end located closer to the terminal end than the one end and connected to the upper wire; and a touch sensor mounted on at least one of the one end and the other end of the torque spring.

The first driving means and the second driving means are upright motors, and the first wire and the second wire are wound on a pulley. The motors and the pulley are engaged by a bevel gear.

The terminal end steering device includes a joystick for controlling a forward operation of the first driving means and the second driving means.

According to still another aspect of the present disclosure, there is provided an endoscope including the above-described terminal end steering device.

### Effects of the Invention

According to the present disclosure, an intelligent portable device loaded with an endoscope driving application is used as an image processing device of an endoscope system. Thus, firstly, the endoscope system is very easy to carry about. Secondly, the endoscope system is very cheap. Thirdly, an intelligent portable device which anyone carries about nowadays can be used as a component of the endoscope system.

Further, according to the present disclosure, a physical and electrical connection/disconnection between an endoscope and the intelligent portable device can be easily made. Thus, it is easy to assemble and disassemble the endoscope system.

Furthermore, according to the present disclosure, the intelligent portable device can supply power required to operate the components provided within the endoscope. Thus, it is not necessary to separately supply power to the endoscope.

With the above-described effects, the present disclosure can perform all of a function to display a current image captured by a camera of the endoscope on a touch screen of the intelligent portable device, a function to zoom in to or out from the current image, a function to adjust an angle of the camera, a function to generate a marker in the current image, a function to capture and store the current image including the marker, a function to compare the current image and a stored image, a function to turn on or off a lighting device of the endoscope, and a function to record the voice of an endoscope user.

Further, the endoscope user can readily use these functions through a touch or drag on the touch screen.

Furthermore, according to the present disclosure, it is possible to perform an operation of marking a defect, which is found during an inspection of an inspection target, in real time without a physical marker or a mechanical/electronic means therefor, and also possible to readily perform this operation through a touch on the touch screen.

Moreover, according to the present disclosure, the number and sequence of defects found during an inspection can be easily checked after the inspection.

Further, according to the present disclosure, a past inspection image and a current inspection image of the inspection target are compared on the touch screen. Thus, firstly, a past defect site in the inspection target can be easily found during a current inspection. Secondly, a current status of the past defect can be easily checked.

Furthermore, according to the present disclosure, it is possible to perform an operation of comparing a current inspection image with each of past inspection images of a certain inspection target, and also possible to readily perform this operation through a touch on the touch screen.

Moreover, the present disclosure can be applied to a general endoscope system for which it is difficult to include a physical marker and a mechanical/electronic means therefor, and makes it easy to carry about the general endoscope system, but does not need a separate means for mounting an intelligent device of the general endoscope system thereon.

Further, according to the present disclosure, while a terminal end is bent, a wire on the opposite side to a wire pulling the terminal end is automatically unwound. Thus, the terminal end can be smoothly bent.

Furthermore, according to the present disclosure, a posture of the bent terminal end can be maintained as long as a user does not perform a separate operation.

Moreover, according to the present disclosure, a switching means is cheap, and, thus, it is suitable to be used in a disposable endoscope.

Further, according to the present disclosure, driving means configured to pull or unwind wires connected to the terminal end can be stood upright. Thus, an area for the driving means is not increased in size.

Furthermore, according to the present disclosure, a joystick is provided. Thus, a bending direction of the terminal end can be easily adjusted.

### BRIEF DESCRIPTION OF THE DRAWINGS

FIG. 1 is a configuration diagram illustrating an inspection system according to the present disclosure;

FIG. 2 illustrates a touch screen of a portable endoscope system;

FIG. 3 is a perspective view illustrating a portable endoscope system according to the present disclosure;

FIG. 4 is a perspective view illustrating the inside of an endoscope head of the portable endoscope system illustrated in FIG. 3;

FIG. 5 is a perspective view illustrating a terminal end steering device according to the present disclosure;

FIG. 6 is an enlarged view illustrating a switching means of the terminal end steering device illustrated in FIG. 5; and

FIG. 7 illustrates a modification example of the switching means illustrated in FIG. 6.

### MODE FOR CARRYING OUT THE INVENTION

Hereinafter, embodiments of the present disclosure will be described in detail with reference to the accompanying drawings so that the present disclosure may be readily implemented by those skilled in the art. However, it is to be noted that the present disclosure is not limited to the embodiments but can be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element. Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Terms and words used in the present specification and claims are not to be construed as a general or dictionary meaning, but are to be construed to meaning and concepts meeting the technical ideas of the present disclosure based on a principle that the inventors can appropriately define the concepts of terms in order to describe their own inventions in the best mode.

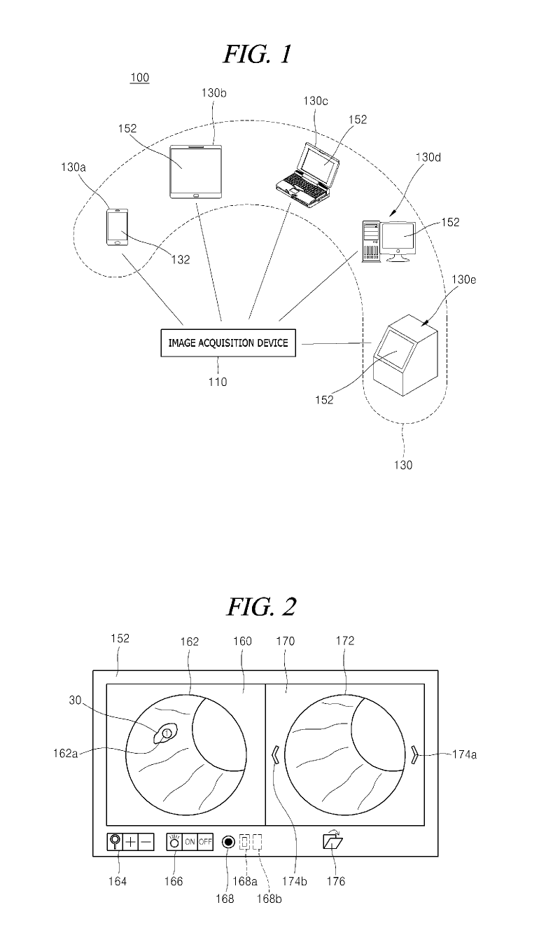

According to the present disclosure, an inspection system 100 capable of marking a defect in real time may include an image acquisition device 110 and an intelligent device 130 as illustrated in FIG. 1.

The image acquisition device 110 may acquire a current inspection image 162 (see FIG. 2) of an inspection target while the presence or absence of a defect in the inspection target is inspected. The image acquisition device 110 may be applied to any field and may have any shape as long as it has the above-described function. For example, the image acquisition device 110 may include a medical device for acquiring an image of a human body while the human body is inspected, a veterinary device for acquiring an image of an animal while the animal is inspected, an architectural device for acquiring an image of a building while the building is inspected, or an industrial device for acquiring an image of various industrial apparatuses or products while the industrial apparatuses or products are inspected.

The intelligent device 130 includes a touch screen 152 and is loaded with a predetermined program, and may perform an operation of installing and executing the program. Examples of the intelligent device 130 may include a smart phone 130a or a smart pad 130b. Further, if a notebook computer 130c, a desktop computer 130d, or a computer 130e of another type includes the touch screen 152, they may be used as the intelligent device 130.

The touch screen 152 of the intelligent device 130 may be of any one of electrostatic type and pressure type. However, if the touch screen 152 is of electrostatic type and an inspector wears sanitary gloves or the like, a touch pen needs to be used or electrostatic touch gloves need to be used as sanitary gloves.

After the image acquisition device 110 and the intelligent device 130 are electrically connected and the program loaded in the intelligent device 130 is executed, the intelligent device 130 may display the content as shown in FIG. 2 on the touch screen 152.

The intelligent device 130 may allocate a current image sector 160 on the touch screen 152 and display a current inspection image 162 being continuously received from the image acquisition device 110 on the current image sector 160. The inspector may inspect the inspection target while watching the current inspection image 162 on the touch screen 152.

Further, the intelligent device 130 may display a file open icon 176 together with the current inspection image 162 on the touch screen 152. If the inspector touches the file open icon 176 to find a desired folder and touches any one past inspection image stored in the folder, the intelligent device 130 may display the past inspection image 172 together with the current inspection image 162 on a stored image sector 170. In this case, the inspector can compare the past inspection image 172 and the current inspection image of the inspection target and thus easily find a past defect site in the inspection target during a current inspection and also easily check a current status of the past defect.

Furthermore, the intelligent device 130 may display page turn icons 174a and 174b together with the past inspection image 172 on the touch screen. If a next icon 174a of the page turn icons 174a and 174b is touched by the inspector, the intelligent device 130 may display a next past inspection image, which is stored in the folder, on the stored image sector 170. If a previous icon 174b is touched by the inspector, the intelligent device 130 may display a previous past inspection image, which is stored in the folder, on the stored image sector 170. In this case, it is possible to easily display multiple past inspection images of a certain inspection target on the stored image sector 170.

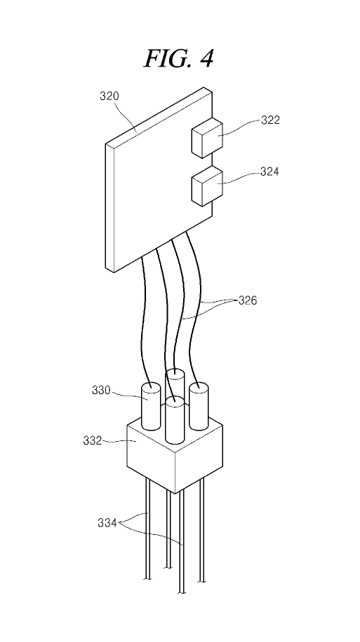

Further, the intelligent device 130 may display a zoom-in/out icon 164 for zooming in to and out from the current inspection image 162 together with the current inspection image 162 on the touch screen 152. If the zoom-in/out icon 164 is touched, the intelligent device 130 may transmit a zoom-in signal to a controller (320 in FIG. 4) of the image acquisition device 110 and the controller may control the camera to zoom in to the current inspection image 162. On the other hand, if the zoom-out icon is touched, the intelligent device 130 may transmit a zoom-out signal to the controller and the controller may control the camera to zoom out the current inspection image 162.

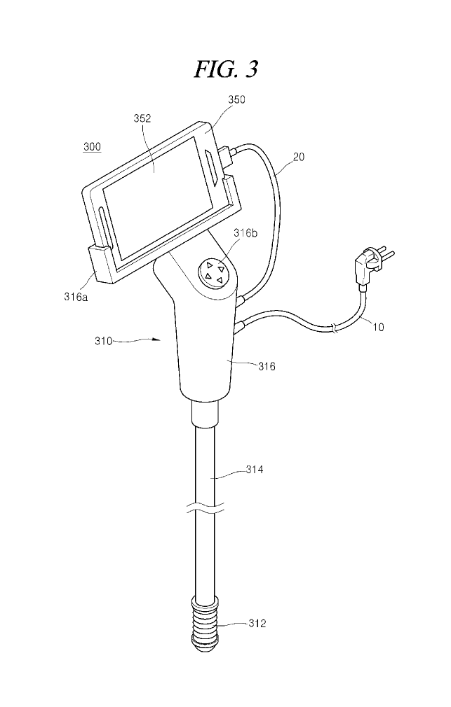

Furthermore, the intelligent device 130 may display a light-on/off icon 166 together with the current inspection image 162 on the touch screen 152. If the light-on icon is touched, the intelligent device 130 may transmit a light-on signal to the controller and the controller may control a lighting device at an endoscope terminal end (312 in FIG. 3) to be turned on. On the other hand, if the light-off icon is touched, the intelligent device 130 may transmit a light-off signal to the controller and the controller may control the lighting device to be turned off.

The intelligent device 130 may display a slide bar (not illustrated) for adjusting brightness of the lighting device on the touch screen 152. In this case, the brightness of the lighting device may be adjusted by dragging the slide bar. Otherwise, a separate button (not illustrated) may be provided at a head (316 in FIG. 3) and the brightness of the lighting device may be adjusted by manipulating the button (not illustrated).

Further, the intelligent device 130 may display a record-on icon 168 and record-off icons 168a and 168b together with the current inspection image 162 on the touch screen 152. If the record-on icon 168 is touched, the intelligent device 130 may operate a recorder included therein to record the voice of an endoscope operator and store the voice in a memory. If the record-off icons 168a and 168b are touched, the intelligent device 130 may stop recording the voice of the endoscope operator. The record-off icons 168a and 168b may include a pause icon 168a and a stop icon 168b.

Furthermore, the intelligent device 130 may generate a marker 162a on the current inspection image 162 and capture and store the current inspection image 162 including the marker 162a. This process may be performed by an instantaneous touch of a user on the current inspection image 162. For example, if the inspector finds a defect 30 from the current inspection image 162 during an inspection and instantaneously touches the defect 30, the intelligent device 130 may indicate the instantaneously touched site with the marker 162a and capture the current inspection image 162 including the marker 162a and then store the current inspection image 162 in its memory (not illustrated). Therefore, in the inspection system 100 according to the present disclosure, a defect can be marked in real time without a physical marker, and, thus, mechanical and electronic means for the physical marker are not needed.

Otherwise, if the defect 30 is instantaneously touched, the intelligent device 130 may display a capture icon (not illustrated) together with the marker 162a, and if the capture icon is touched, the intelligent device 130 may capture and store the current inspection image 162 including the marker 162a. In this case, the inspector may directly determine whether or not to capture and store the current inspection image 162 including the marker 162a.

Further, the intelligent device 130 may assign an identifier to the marker 162a according to the sequence of generated markers 162a and then display the marker 162a on the touch screen 152. For example, if the inspector touches a defect which is first found during an inspection, the intelligent device 130 assigns No. 1 to the first found defect and displays the defect on the touch screen, and if the inspector touches a defect which is subsequently found, the intelligent device 130 assigns No. 2 to this defect to be displayed. In this case, the number and sequence of defects found during an inspection can be easily checked after the inspection. For reference, FIG. 2 illustrates “1” as an example of a numerical identifier. Further, letters, symbols, and the like may be used as identifiers instead of numbers.

Further, when the marker 162a is generated, the intelligent device 130 may display a pop-up window (not illustrated) in which an identifier can be input, on the touch screen 152. In this case, the inspector may directly input an identifier to be assigned to the marker 162a. Further, although not illustrated, any identifier (e.g., color, shape, date, and time) other than the number may be assigned to the marker 162a by the intelligent device 130.

Meanwhile, as a general medical or industrial endoscope, the image acquisition device 110 may be provided and used in the form of a tube to be inserted into an inspection target without a need to disassemble or incise the inspection target. However, under the current circumstances, if an inspector finds a defect from an image captured by a camera at a terminal end of the general endoscope, the inspector stores the image and marks the defect while checking the stored image after the inspection. This is because it is very difficult to provide mechanical and electronic means for a physical marker in the narrow tube-shaped general endoscope.

However, if the general endoscope is used as the image acquisition device 110 of the inspection system 100 according to the present disclosure, while an inspection target is inspected, a defect can be marked in real time. In this case, a physical marker or a mechanical means and an electronic means for the physical marker are not needed. Therefore, the inspection system 100 according to the present disclosure is very effective when the image acquisition device 110 has a tube shape to be inserted into an inspection target without a need to disassemble or incise the inspection target like a general endoscope.

The smart phone 130a or the smart pad 130b may be used as the intelligent device 130. In this case, the endoscope used as the image acquisition device 110 of the inspection system 100 and the smart phone 130a or the smart pad 130b used as the intelligent device 130 are all easy to carry about. Thus, the inspection system 100 is easy to carry about. Further, the smart phone 130a or the smart pad 130b which most people carry about nowadays can be used as a component of the inspection system 100.

If the inspection system 100 is an endoscope system as described above, the endoscope as the image acquisition device 110 may include a cradle 316a for mounting the smart phone 130a or the smart pad 130b as the intelligent device 130 (see FIG. 3). The endoscope is inserted into the inspection target through a manual operation of the inspector, and a camera provided at a terminal end of the endoscope is steered through a manual operation of the inspector. Therefore, it is difficult for the inspector to perform the inspection while holding the smart phone 130a or the smart pad 130b on one hand. Accordingly, the smart phone 130a or the smart pad 130b needs to be mounted within touch of the inspector. However, if the cradle is provided in the endoscope as described above, a separate means for mounting the smart phone 130a or the smart pad 130b is not needed.

Herein, the cradle 316a may have various shapes. For example, the cradle 316a may be of sliding type or tong type. In the former case, the smart phone 130a or the smart pad 130b may be slidably mounted on the cradle 316a, and in the latter case, the smart phone 130a or the smart pad 130b may be held by the cradle 316a.

Hereinafter, a portable endoscope system 300 as one example of the inspection system 100 according to the present disclosure will be described. The portable endoscope system 300 according to the present disclosure may be used not only for medical purpose but also for industrial purpose. However, in the following, an example where the portable endoscope system 300 is used for medical purpose will be described for convenience in explanation.

The portable endoscope system 300 may include an endoscope 310 and an intelligent portable device 350 as illustrated in FIG. 3. The endoscope 310 is one example of the image acquisition device 110 illustrated in FIG. 1 and the intelligent portable device 350 is one example of the intelligent device 130 illustrated in FIG. 1.

The endoscope 310 may include a terminal end 312, a head 316, and a central part 314. The terminal end 312 includes multiple joints and thus can be bent in various directions. Although not illustrated, a camera configured to photograph around the terminal end 312 and a lighting device configured to irradiate a light to a site to be photographed by the camera may be further provided at the terminal end 312. Further, the endoscope 310 may include bellows configured to cover the joints of the terminal end 312. The head 316 is located on the opposite side to the terminal end 312, and the central part 314 may connect the terminal end 312 and the head 316.

The cradle 316a for mounting the intelligent portable device 350 may be provided at the head 316 of the endoscope 310. FIG. 3 illustrates the cradle 316a of sliding type. However, the cradle 316a of tong type capable of holding the intelligent portable device 350 may be provided.

The head 316 of the endoscope 310 may include an actuator 330 and a controller 320 as illustrated in FIG. 4. The actuator 330 may include a pair of motors for bending the terminal end 312 of the endoscope 310 in left and right directions and another pair of motors for ending the terminal end 312 in forward and backward directions. Further, the actuator 330 is combined with a gear box 332. The gear box 332 may include pulleys (not illustrated) of the same number as the motors and bevel gears (not illustrated) configured to connect the motors and the pulleys. On the pulleys (not illustrated), wires 334 are wound. These wires 334 are extended to the terminal end 312 through the central part 314 of the endoscope 310 and configured to pull or loosen the terminal end 312 by being wound on or unwound from the pulleys (not illustrated). Meanwhile, the actuator 330 may have any other shape as long as it can bend the terminal end 312 of the endoscope 310 in every direction.

The controller 320 is configured to control the actuator 330 and the camera and the lighting device at the terminal end 312 of the endoscope 310. In order to control the actuator 330, the controller 320 is connected to the actuator 330 through an electrical wire 326. In order to control the camera and the lighting device at the terminal end 312 of the endoscope 310, the controller 320 is connected to them through electrical wires (not illustrated). Further, the controller 320 may include a USB port 322. If the USB port 322 and the intelligent portable device 350 are connected through a USB cable 20, the controller 320 and the intelligent portable device 350 are electrically connected to each other. Furthermore, the controller 320 may include a power supply port 324 for a connection to a power supply line 10. Power supplied to the power supply port 324 through the power supply line 10 is used to operate the controller 320, the actuator 330, the camera, and the lighting device. A printed circuit board may be used as the controller 320.

Otherwise, the USB port 322 may be provided at the bottom of the cradle 316a and exposed to the outside. In this case, when the intelligent portable device 350 is mounted on the cradle 316a, an electrical connection between the intelligent portable device 350 and the controller 320 is directly made.

The intelligent portable device 350 includes a touch screen 352 and is loaded with an endoscope driving application, and may install and execute the endoscope driving application. Further, the intelligent portable device 350 may have a wireless communication function to wirelessly download the endoscope driving application. A smart phone, a smart pad, or the like may be used as the intelligent portable device 350.

The touch screen 352 may be of any one of electrostatic type and pressure type.

If the endoscope 310 is electrically connected to the intelligent portable device 350 and the endoscope driving application is executed, the intelligent portable device 350 supplies power to the controller 320. The power may be used to operate the controller 320, the actuator 330, the camera, and the lighting device. Even if the intelligent portable device 350 is used as a power supply source, the controller 320 includes the power supply port 324 as described above. If a battery charge amount in the intelligent portable device 350 is not sufficient, power supplied through the power supply port 324 may be used.

Further, if the endoscope 310 is electrically connected to the intelligent portable device 350 and the endoscope driving application is executed, the intelligent portable device 350 displays the content as shown in FIG. 2 on the touch screen 352. The intelligent portable device 350 may allocate the current image sector 160 on the touch screen 352 and display the current inspection image 162 being captured by the camera on the current image sector 160. In order to do so, the controller 320 may continuously receive the current inspection image 162 from the camera and transmit the current inspection image 162 to the intelligent portable device 350.

Furthermore, the intelligent portable device 350 may adjust a bending direction of the terminal end 312 of the endoscope 310 through a drag (scratching the current inspection image being touched) of the current inspection image 162. For example, if the current inspection image 162 is dragged to left, the intelligent portable device 350 may transmit a left bending signal to the controller 320 and the controller 320 may control the actuator 330 to bend the terminal end 312 of the endoscope 310 to left. If the current inspection image162 is dragged to right, the intelligent portable device 350 may transmit a right bending signal to the controller 320 and the controller 320 may control the actuator 330 to bend the terminal end 312 of the endoscope 310 to right. Likewise, the terminal end 312 of the endoscope 310 can be bent forwards and backwards.

Meanwhile, a joystick 316b is provided at the head 316 of the endoscope 310 as illustrated in FIG. 3, and includes a forward, backward, left, right input button. If an endoscope operator pushes the input button, a signal corresponding thereto may be transmitted to the controller 320 and the controller 320 may control the actuator 330 in response to the received signal. The endoscope operator may adjust a bending direction of the terminal end 312 of the endoscope 310 by dragging the touch screen 352 or using the joystick 316b.

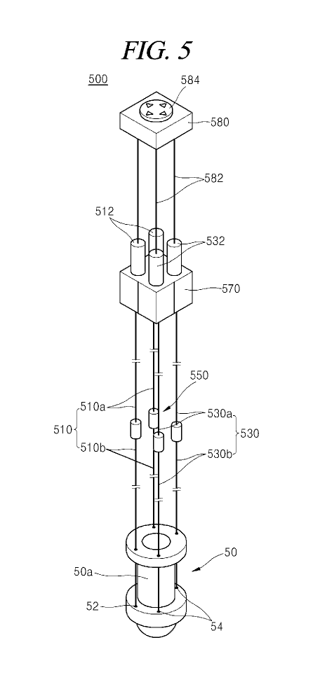

FIG. 5 is a perspective view illustrating a terminal end steering device according to the present disclosure.

A terminal end steering device 500 according to the present disclosure may include both a configuration for adjusting a bending direction of a terminal end 50 to left and right directions and a configuration for adjusting the bending direction to forward and backward directions. The configuration for forward and backward bending of the terminal end 50 is identical to the configuration for left and right bending of the terminal end 50. Therefore, in the following, any one of them will be described.

The terminal end steering device 500 may include a first wire 510, a second wire 530, a switching means 550, and a control unit (not illustrated, but, for example, 320 in FIG. 4). Herein, the terminal end 50 includes a joint part 50a including multiple joints and can be bent in various directions by the joint part 50a. Although not illustrated, a camera configured to photograph around the terminal end 50 and a lighting device configured to irradiate a light to a site to be photographed by the camera may be further included in the terminal end 50.

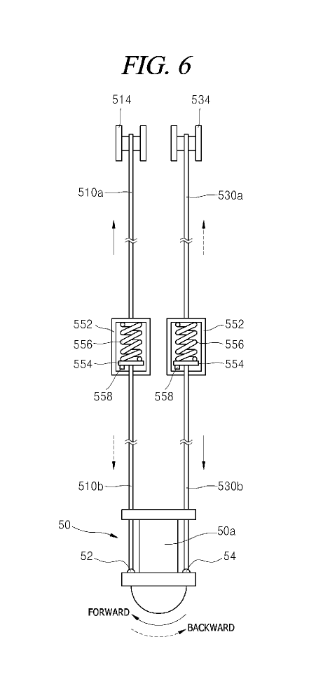

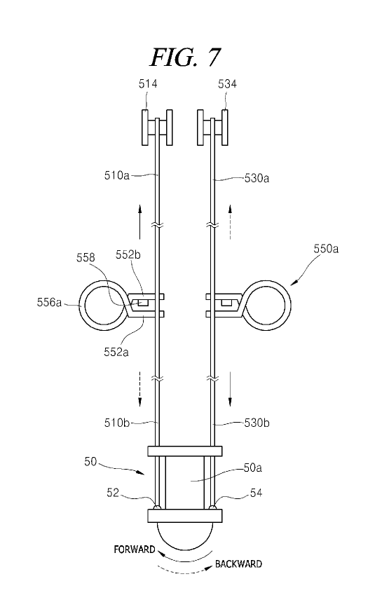

The first wire 510 may include an upper wire 510a and a lower wire 510b separated from each other. The lower wire 510b is located closer to the terminal end 50 than the upper wire 510a, and includes an upper end combined with the switching means 550 and a lower end connected to one side 52 of the terminal end 50. The upper wire 510a is located on the opposite side to the lower wire 510b with reference to the switching means 550, and includes a lower end combined with the switching means 550 and an upper end wound on a first pulley (514 in FIG. 6). If the first pulley 514 is rotated in a forward direction, the first wire 510 formed as such is wound on the first pulley 514 to pull the one side 52 of the terminal end 50. In this case, the terminal end 50 is bent in the forward direction.

The second wire 530 may include an upper wire 530a and a lower wire 530b separated from each other. The lower wire 530b is located closer to the terminal end 50 than the upper wire 530a, and includes an upper end combined with the switching means 550 and a lower end connected to another side 54 spaced 180 degrees away from the one side 52 of the terminal end 50. The upper wire 530a is located on the opposite side to the lower wire 530b with reference to the switching means 550, and includes a lower end combined with the switching means 550 and an upper end wound on a second pulley (534 in FIG. 6). If the second pulley 534 is rotated in a forward direction, the second wire 530 formed as such is wound on the second pulley 514 to pull the other side 54 of the terminal end 50. In this case, the terminal end 50 is bent in a backward direction.

The first pulley 514 and the second pulley 534 on which the first wire 510 and the second wire 534 are respectively wound may be accommodated in a gear box 570. Further, the pulleys 514 and 534 may be respectively connected to a first motor 512 serving as a first driving means and a second motor 532 serving as a second driving means through a bevel gear (not illustrated) accommodated in the gear box 570. Therefore, if the motors 512 and 532 operate in the forward direction, the pulleys 514 and 534 are also rotated in the forward direction.

If the pulleys 514 and 534 and the motors 512 and 532 are connected as such, the motors 512 and 532 may be stood upright in parallel with each other. Thus, the head 316 of the endoscope 310 in which the motors 512 and 532 are accommodated is not increased in size.

Meanwhile, if the first wire 510 pulls the one side 52 of the terminal end 50 but the second wire 540 is not unwound from the second pulley 534, the terminal end 50 cannot be bent in the forward direction. If the second wire 530 pulls the other side 54 of the terminal end 50 but the first wire 510 is not unwound from the first pulley 514, the terminal end 50 cannot be bent in the backward direction. Therefore, if the first pulley 514 is rotated in the forward direction, the second pulley 534 is required to be rotated in the backward direction, and if the second pulley 534 is rotated in the forward direction, the first pulley 514 is required to be rotated in the backward direction. In the present disclosure, such requirements are satisfied by the switching means 550 and the control unit (not illustrated).

The switching means 550 is provided at each of the first wire 510 and the second wire 530. As illustrated in FIG. 6, the switching means 550 may include a case 552, a sliding plate 554, a tensile spring 556, and a touch sensor 558.

The case 552 may include an upper surface combined with the lower end of the upper wire 510a, 530a and a lower surface through which the lower wire 510b, 530b penetrates. The sliding plate 554 is slidably accommodated in an internal space of the case 552 and may be combined with the upper end of the lower wire 510b, 530b. The tensile spring 556 is accommodated in the internal space of the case 552 and may include both ends respectively combined with the upper surface of the case 552 and the sliding plate 554. The tensile spring 556 may exert elastic force in a direction in which the upper surface of the case 552 and the sliding plate 554 get closer to each other. The touch sensor 558 is provided at one surface of the sliding plate 554 facing the lower surface of the case 552, and when applied with pressure by the lower surface of the case 552, the touch sensor 558 may output an electrical signal. Otherwise, the touch sensor 558 may be provided at the lower surface of the case 552. In this case, when applied with pressure by the sliding plate 554, the touch sensor 558 may output an electrical signal.

If the first driving means 512 operates in the forward direction, the upper wire 510a of the first wire 510 is pulled toward the first pulley 514. In this case, the case 552 overcomes the elastic force of the tensile spring 556 and applies pressure to the touch sensor 558 at the sliding plate 554 combined with the lower wire 510b of the first wire 510. If the case 552 applies pressure to the touch sensor 558, the lower wire 510b of the first wire 510 is pulled toward the first pulley 514 and the lower wire 530b of the second wire 530 is pulled toward the terminal end 50. In this case, the second driving means 532 is stopped. Therefore, the sliding plate 554 combined with the lower wire 530b overcomes the elastic force of the tensile spring 556, and, thus, the touch sensor 558 at the sliding plate 554 combined with the lower wire 530b is applied with pressure by the case 552 combined with the upper wire 530a of the second wire 530.

As such, when the first driving means 512 starts operating in the forward direction, all the touch sensors 558 of the switching means 550 respectively provided on the wires 510 and 530 are applied with pressure and output electrical signals. Further, if the electrical signals are output, the control unit may operate the second driving means 532, which does not operate in the forward direction, of the first driving means 512 and the second driving means 532 in the backward direction. Further, if the second driving means 532 operates in the backward direction, the second wire 530 is unwound from the second pulley 534, and, thus, the terminal end 50 can be bent in the forward direction.

If the forward operation of the first driving means 512 is stopped and the forward bending of the terminal end 50 is stopped accordingly, the pressures applied to the touch sensors 558 are relieved by the elastic force of the tensile springs 556. Thus, the backward operation of the second driving means 532 is also stopped and the unwinding of the second wire 530 is stopped. In this case, a bent posture of the terminal end 50 is maintained until the driving means 512 and 532 operate again.

The above-described process performed during a forward bending of the terminal end 50 may be also performed in the same manner during a backward bending of the terminal end 50.

The control unit (e.g., 320 in FIG. 4) which performs the above-described function may be configured as a printed circuit board and accommodated in a control box 580. Further, the control unit may control the driving means 512 and 532 as being connected to the driving means 512 and 532 through electrical wires 582 and also receive an electrical signal output from the touch sensor 558 as being connected to the touch sensor 558 through other electrical wires (not illustrated).

Meanwhile, the switching means 550 may be substituted with a switching means 550a illustrated in FIG. 7. The switching means 550a may include a torque spring 556a and the touch sensor 558. One end 552b of the torque spring 556a may be combined with the upper wire 510a, 530a, and another end 552a located closer to the terminal end 50 than the one end 552b may be combined with the lower wire 510b, 530b. The torque spring 556a has elastic force in a direction in which the one end 552b and the other end 552a get away from each other. The touch sensor 558 may be provided at any one of the both ends 552a and 552b of the torque spring 556a. An operation of the switching means 550a is identical to the above-described operation of the switching means 550. Thus, explanation thereof will be omitted.

The terminal end steering device 500 may include a button-type joystick 584. The button-type joystick 584 includes a left, right, forward, backward input button, and may be provided on the control box 580 in which the control unit is accommodated. If a user pushes the left, right, forward, backward input button of the joystick 584, input information may be transmitted to the control unit and the control unit may control forward operations of the driving means 512 and 532 in response to the received information. As described above, backward operations of the driving means 512 and 532 may be automatically performed by the switching mean 550, 550a and the control unit. The button-type joystick 584 may be substituted with a stick-type joystick (not illustrated).

The terminal end steering device 500 can be used in any device of which a bendable terminal end 50 needs to be adjusted in bending direction. For example, the terminal end steering device 500 may be provided in the endoscope 310 including the head 316, the terminal end 312, and the central part 314 connecting the head 316 and the terminal end 312, as illustrated in FIG. 3. In this case, the control box 580, the driving means 512 and 532, and the gear box 570 of the terminal end steering device 500 are provided within the head 316 of the endoscope 310 such that the joystick 584 is exposed, and the wires 510 and 530 and the switching mean 550, 550a may be provided within the central part 314.

The above description of the present disclosure is provided for the purpose of illustration, and it would be understood by those skilled in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

We claim:

1. A portable endoscope system comprising:
an endoscope including a terminal end at which a camera and a lighting device are provided, a head provided on the opposite side to the terminal end, an actuator provided within the head to bend the terminal end, and a controller provided within the head to control the actuator, the camera, and the lighting device; and
an intelligent portable device including a touch screen, loaded with an endoscope driving application, and provided to be electrically connected to the controller.

2. The portable endoscope system of claim 1,
wherein at the head of the endoscope, a cradle for mounting the intelligent portable device thereon is provided.

3. The portable endoscope system of claim 1,
wherein after the endoscope and the intelligent portable device are electrically connected to each other and the endoscope driving application is executed, the intelligent portable device supplies the controller with power required to operate the actuator, the camera, the lighting device, and the controller.

4. The portable endoscope system of claim 1,
wherein after the endoscope and the intelligent portable device are electrically connected to each other and the endoscope driving application is executed, the intelligent portable device receives a current image being captured by the camera from the controller and displays the current image on the touch screen.

5. The portable endoscope system of claim 4,
wherein the intelligent portable device displays a zoom-in/out icon for zooming in to and out from the current image together with the current image on the touch screen, and if the zoom-in/out icon is touched, the intelligent portable device transmits a zoom-in/out signal corresponding thereto to the controller.

6. The portable endoscope system of claim 4,
wherein if the current image is dragged, the intelligent portable device transmits a terminal bending signal, which instructs the controller to bend the terminal end of the endoscope in a dragged direction, to the controller.

7. The portable endoscope system of claim 4,
wherein if any one point in the current image is touched, the intelligent portable device generates a marker at the touched point and then captures the current image including the marker and stores the current image in its memory.

8. The portable endoscope system of claim 4,
wherein the intelligent portable device displays a stored image read from a folder specified by a user together with the current image on the touch screen.

9. The portable endoscope system of claim 8,
wherein the intelligent portable device displays a page turn icon together with the stored image on the touch screen, and if the page turn icon is touched, the intelligent portable device displays another stored image in the folder together with the current image on the touch screen.

10. The portable endoscope system of claim 4,
wherein the intelligent portable device displays an on/off icon for the lighting device together with the current image on the touch screen, and if the on/off icon is touched, the intelligent portable device transmits an on/off signal corresponding thereto to the controller.

11. The portable endoscope system of claim 4,
wherein the intelligent portable device displays an on/off icon for a recorder included therein together with the current image on the touch screen, and if the on/off icon is touched, the intelligent portable device operates or stops the recorder.
